(19) United States
(12) Patent Application Publication
(10) Pub. No.: US 2026/0223821 A1
(43) Pub. Date: Aug. 6, 2026

(54) PRECISION DELIVERY OF ORAL AND TOPICAL SUBSTANCES TO ANIMAL POPULATIONS

(71) Applicant: University of Idaho, Moscow, ID (US)

(72) Inventors: Scott Nuismer, Troy, ID (US); Luke Sheneman, Moscow, ID (US); Michael Maughan, Troy, ID (US)

(73) Assignee: University of Idaho, Moscow, ID (US)

(21) Appl. No.: 19/149,485

(22) PCT Filed: Jan. 17, 2024
(86) PCT No.: PCT/US2024/011846

§ 371 Date: Jul. 18, 2025

Related U.S. Application Data
(60) Provisional application No. 63/439,921, filed on Jan. 19, 2023.

(51) Int. Cl. A61D 7/00; A01K 11/00; G06V 20/52

(52) U.S. Cl. CPC A01K 29/005; A01K 13/003; A01M 25/00; A01M 29/00; A61D 7/00; G06V 20/52

(57) ABSTRACT

An administration apparatus is disclosed. The administration apparatus according to the present disclosure can include one or more imaging devices, a computer configured to receive image data from the one or more imaging devices, a floorplate operatively coupled to the computer, and one or more reservoirs each coupled to a respective administration device configured to administer one or more attractants and/or substances when directed by the computer. The computer can be configured to identify whether the image data shows a target subject or a non-target subject. If the computer identifies a target subject the apparatus can administer a substance, and if the computer identifies a non-target subject the apparatus can provide a deterrent such that the non-target subject is discouraged from remaining near the apparatus.

### CROSS REFERENCE TO RELATED APPLICATION

The present application claims the benefit of and priority to the earlier filing date of U.S. Provisional Application 63/439,921, filed Jan. 19, 2023, which application is incorporated herein by reference in its entirety.

### FIELD

The present disclosure concerns apparatuses for attracting, identifying, measuring, recording, and administering one or more substances to subject wild animal populations.

### BACKGROUND

Current methods and apparatuses for delivering oral and topical substances to wild animal populations are primitive and imprecise. As a consequence, efforts to vaccinate, medicate, mark/tag, and/or poison wild animal populations rely on labor intensive capture and manipulation of individual animals or indiscriminate and wasteful mass distribution of medicaments and/or poisons. For example, to reduce the risk that rabid raccoons infect humans or domestic animals, the USDA distributes millions of vaccine-laced baits each year via vehicles and aircraft. Although this program is effective at reducing the risk of rabies, much of the distributed vaccine is wasted when it is (a) never consumed, (b) consumed by animals other than the targeted species, and/or (c) hoarded by individual animals.

Inefficiencies also arise in situations such as scientific research where it is often important to mark/tag individual animals so that they can be identified upon recapture. Current practice achieves this by capturing animals using traditional traps which are then regularly monitored and/or checked by trained researchers. When the targeted animal species is captured, it is then marked and released. This practice involves trapping many off-target animals and large amounts of skilled human labor. Similar challenges arise with efforts to medicate or poison specific animal species or individual animals.

Accordingly, there is a continuing need for improved apparatuses and methods for delivery of substances to wild animal populations.

### SUMMARY

Described herein are apparatuses for attracting, identifying, measuring, recording, and administering one or more substances to subject wild animal populations.

An administration apparatus can generally comprise a computer configured to receive data related to a subject adjacent the computer, the computer configured to identify whether the data indicates the presence of either a target subject or a non-target subject, and an administration apparatus configured to administer an attractant and/or a substance when directed by the computer. In addition to these components, an administration device can further comprise one or more of the components disclosed herein.

In some examples, an administration apparatus can comprise a floorplate operatively coupled to the computer.

In some examples, an administration apparatus can comprise a reservoir coupled to the administration device.

In some examples, the apparatus is configured to administer a substance to the target subject when the computer identifies the target subject.

In some examples, the apparatus is configured to provide a deterrent to compel the non-target subject to move away from the apparatus when the computer identifies the non-target subject.

In some examples, the apparatus further comprises one or more imaging devices configured to provide image data to the computer. In some examples, the computer identifies whether the subject is a target subject or a non-target subject based on the image data by using a neural network.

In some examples, the apparatus comprises a housing defining a chamber having an opening.

In some examples, the apparatus is configured to administer an attractant at a selected time interval.

In some examples, an administration apparatus, comprises a housing defining a chamber having an opening, an imaging device configured to provide image data showing an interior of the chamber, a computer configured to receive the image data from the imaging device, and a reservoir coupled to an administration device configured to administer an attractant and/or a substance to the interior of the chamber when directed by the computer. The computer can be configured to identify whether the image data shows a target subject or a non-target subject within the chamber. The administration apparatus can be configured to (1) administer the attractant at a selected time interval; (ii) administer the substance when the computer identifies the target subject; and (iii) provide a deterrent such that the non-target subject is compelled to move away from the apparatus when the computer identifies a non-target subject.

In some examples, a method comprises attracting a subject to an administration apparatus using an attractant; using a computer to identify whether the subject is a target subject or a non-target subject based on data related to the subject; deterring the subject from approaching the administration apparatus using a deterrent if the subject is a non-target subject; and administering a substance to the target subject using an administration device if the subject is a target subject, wherein each administration device is coupled to a respective reservoir.

In some examples, an administration apparatus and/or a method comprises one or more of the components or acts recited in Examples 1-20 herein.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a schematic view of an administration apparatus, according to one example of the present disclosure.

FIG. 2 is a perspective view of the administration apparatus of FIG. 1.

FIG. 3 is a flow-chart illustrating an exemplary method of using the administration apparatus of FIG. 1.

FIG. 4 is a schematic diagram of an exemplary computing environment.

### DETAILED DESCRIPTION

### General Considerations and Definitions

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the subject. As used herein, the term “distal” refers to a position, direction, or portion of a device that is further away from the subject. The terms “longitudinal” and “axial” refer to an axis extending in the proximal and distal directions, unless otherwise expressly defined. In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

As used herein, “e.g.” means “for example,” and “i.e.” means “that is.”

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited. As used herein, the term “about” means the listed value still produces the functional result associated with the value, and may be within 10% of the listed value. For example, “about 100 degrees” means at least any value between 90-110 degrees, inclusive.

The term “substance” as used herein encompasses medicaments, identifying markings or tracking tags, and poisons. The term “medicament” as used herein refers to any beneficial substance that may be administered to a subject. Particular examples include, for example and without limitation, medicines, antibiotics, vaccines, therapeutics, hormones, food supplements, oils, vitamins, minerals, etc. The term “identifying markings” as used herein includes substances used as unique identifiers, tracking substances, marking substances (such as paint or dyes), and marking/tracking tags (such as PIT tags), etc. The term “poison” as used herein refers to any detrimental (e.g., toxic and/or hazardous to health, well-being, and/or reproductive capabilities) substance that may be administered to a subject, for example, hydrogen cyanide. In some embodiments, the substances may be in liquid form. In other embodiments, the substances may be in powdered form and may be mixed with one or more liquids/solvents/food items.

The term “subject” as used herein refers to a non-human animal subject, such as a wild animal. The term “animal” can refer to mammals, birds, amphibians, reptiles, etc. For example, animals can include but are not limited to raccoons, opossums, dogs, cats, deer, moose, bats, and rodents such as deer mice, rats, etc.

### Examples of the Disclosed Technology

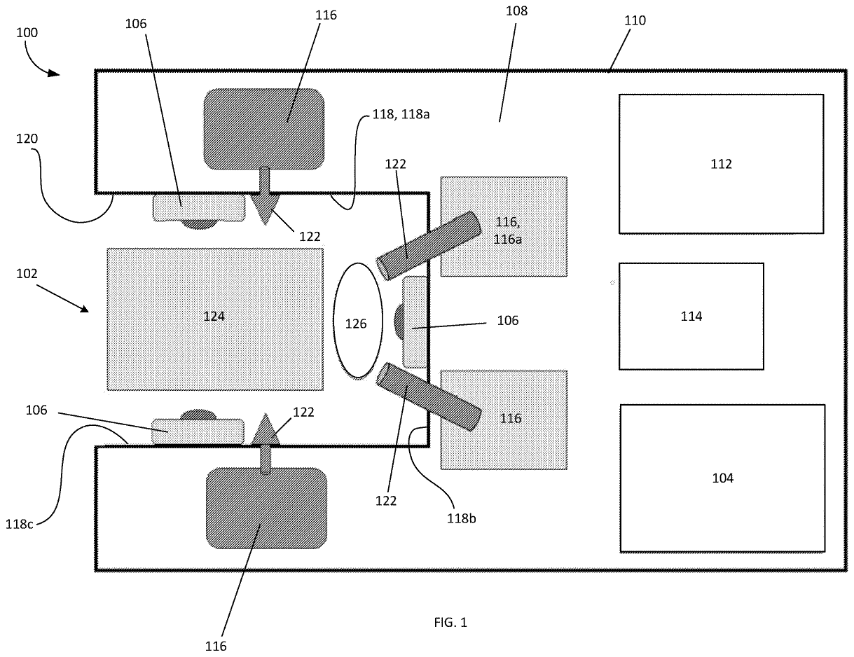

FIG. 1 illustrates an exemplary administration apparatus 100. The apparatus 100 can be generally configured to (1) attract a subject to a chamber 102 within the apparatus 100, (2) identify whether the subject is a target subject and/or a member of a target species of subjects using a computer 104 implementing an algorithm (e.g., an object detection and classification algorithm such as a neural network and/or other image recognition algorithm), (3) record the identity of the subject (e.g., by sending image data to a remote device), (4a) if the subject is identified as a non-target subject and/or species, deterring the subject using a deterrence method to cause the subject to exit the chamber 102, or (4b) if the subject is identified as a target subject and/or a member of a target species of subjects, administering a substance to the target subject.

The exemplary administration apparatuses 100 described herein can be used, for example, to automate vaccination and/or prophylaxis of targeted wildlife species. Typical vaccination methods for wild animal populations rely on indiscriminate bulk distribution of vaccine-laced baits. While this conventional method may work for some species of wild animals, it is ineffective for short-lived animals such as rodents (which can harbor a myriad of human diseases). It is increasingly important to accurately vaccinate target species, as the risk of pathogen spillover into the human population grows in response to climate change and human encroachment on the habitat of wild animals. The apparatuses described herein allow substances to be efficiently and precisely distributed to target subjects automatically.

In addition to providing a mechanism for distributing vaccine-laced edible substances (such as baits), the apparatuses described herein (e.g., apparatus 100) can facilitate the delivery of nascent transferable vaccines that can be applied as a topical solution that self-disseminates to other animals through the process of allogrooming. In other words, the apparatuses can be configured to apply a topical substance to a first target subject, that can then be distributed to one or more additional target subjects through the process of grooming after the first target subject has exited the administration apparatus 100. Because these apparatuses 100 are designed to deliver substances only to the target species and do not allow individual subjects to hoard substances, these apparatuses 100 will vastly reduce the amount of vaccine required to achieve herd immunity.

Further, the apparatuses described herein (e.g., apparatus 100) can advantageously be used to automate subject recapture studies. Known approaches to studying wild animal populations involve capturing individual animals, marking them, then recapturing them later. This technique enables population sizes to be estimated, rates of predation to be quantified, and patterns of movement to be better understood. Current state-of-the-art for mark-recapture studies relies on setting traditional live traps (e.g., Sherman traps) and checking those traps daily for the presence of the target species. When a capture occurs, the subject is marked, which may require the use of anesthesia. Recapture occurs when an already marked subject is found in a trap. This technique is expensive, time-consuming, and stressful for the captured subjects.

The apparatuses described herein advantageously automate the mark-recapture process without ever trapping or detaining the subject, which advantageously reduces stress on the subjects and lowers costs by reducing the amount of skilled labor involved. The apparatuses 100 are placed in the field and the process of marking and ‘recapturing’ is performed automatically, with relevant data recorded and passed on to those conducting the analysis. In addition to the reduction of cost and effort, the described apparatuses also allow for mark-recapture studies to take place in dangerous, hard-to-reach, or hostile environments that would not allow for the repeated manual checking of traps.

The apparatuses can further be used to cull populations of undesirable species. Known approaches to this problem have typically involved mass distribution of poison. However, mass poisoning is indiscriminate and can cause significant mortality in non-target species. The apparatuses described herein allow poison to be precisely distributed only to target undesirable populations of wild animals in the form of edible baits or topical solutions. Because these apparatuses 100 deliver poison only to target species, poisoning efficiency is increased and collateral damage is reduced.

Further, the apparatuses described herein (e.g., apparatus 100) can be used for automated health assessments of wild animal species. Monitoring the health of wild animal populations is essential for conservation, game management, and early identification of infectious disease, among other things. Quantifying the health of living animals generally requires that they be captured, weighed, measured, and potentially sampled for the presence of infectious disease. Known procedures to achieve these steps are labor intensive, costly, and can entail significant risk to the humans performing the studies/testing (e.g., the potential transfer of zoonotic diseases). The apparatuses 100 described herein advantageously overcome these challenges by automating the process of capture and assessment without the need for human interaction or trapping of the target subjects. Instead, these apparatuses 100 use an integrated scale and/or imaging devices connected to a computer 104 that is configured to execute recognition and measurement algorithms.

In some examples, such as the example illustrated in FIGS. 1-2, the apparatus 100 can comprise one or more imaging devices 106 (e.g., cameras) operatively coupled to the computer 104, a main body 108 including a housing 110 that contains a power source 112 and a control unit 114 (which can receive signals from the computer to execute commands such as releasing an attractant, releasing a substance, and/or providing a deterrent), and one or more reservoirs 116 configured to store one or more attractants and/or substances for administration. The computer 104 can be an onboard part of the apparatus 100 (e.g., disposed within the housing 110), or alternatively can be remote to the apparatus 100.

In other examples, the apparatus 100 can not include any imagining devices. In such examples, the computer 104 can determine whether the subject is a target subject or a non-target subject based on the presence or absence of a marking substance, such as a PIT tag.

The power source 112 can be, for example, a battery, a solar panel, a hydrogen fuel cell, AC power from an electrical grid or generator, etc. Combinations of power sources can also be used. In some examples, the power source 112 can be rechargeable. In some examples, the power source can be a wired power source, such as an electrical current provided by an electrical grid. In some examples, the computer 104 can be configured to send an alert to a remote device when the power source is low on power.

The main body 108 can define a recess or chamber 102 that extends into the main body 108. In some examples, the chamber 102 can comprise three walls, a first wall 118a, a second wall 118b, and a third wall 118c. The chamber 102 can further comprise an opening/inlet/entryway 120. In some examples, the opening 120 can comprise a sensor (e.g., a motion-detecting sensor, a heat sensor, a pressure sensor positioned for example in the floor plate, etc.) that is configured to detect the entry of a subject into the chamber 102. In such examples, the computer 104 and/or control unit 114 can be configured to be in a low power “sleep state” (or entirely off) until triggered by the sensor, which turns the computer 104 and/or control unit 114 to a “powered on” state.

The dimensions of the apparatus 100 can be adjusted to accommodate a target subject and/or target species-for example, in some examples the apparatus 100 can be small enough to accommodate rodents within the chamber 102, or in other examples the apparatus 100 can be large enough to accommodate deer and/or moose.

In some examples, such as when the apparatus 100 is sized to accommodate large animals such as deer and/or moose, the apparatus 100 need not include a main body 108 or housing 110 or an enclosed or semi-enclosed chamber 102. For examples, the apparatus 100 can include one or more imaging devices 106 configured to communicate with a remote control unit 114 and remote computer 104 (e.g., using Bluetooth, Wi-Fi, etc.), and one or more administration devices 122, such as spray nozzles, coupled to one or more reservoirs 116 (for example, an overhead spray nozzle similar to a shower). In such examples, the large-sized apparatus 100 can function in the same or similar manner as described herein.

In some examples, the opening 120 can include a door or gate that can be configured to close automatically when a subject is within the chamber 102. In some examples, the gate can be configured to open automatically once administration of one or more substances is complete. In other examples, the gate can be configured to remain closed until manually opened (or remotely triggered) by an operator. This allows the apparatus 100 to be used to capture and hold specific subjects if desired. In some examples, the apparatus 100 can comprise one or more holding chambers configured to retain one or more captured subjects. Such a configuration allows the apparatus 100 to capture multiple target subjects for collection by an operator. In such embodiments, for example, the chamber 102 could lead to a respective chamber of a plurality of holding chambers, and the holding chambers could rotate relative to the chamber 102 such that when a holding chamber is filled with a subject it rotates away from the chamber 102 and a new, empty holding chamber takes its place. In this way, the apparatus can store multiple subjects in multiple holding chambers for collection by an operator.

In some examples, the chamber 102 can further comprise a floor plate 124. In some examples, the floor plate 124 can comprise a scale or other measurement device (e.g., a thermometer, heart rate monitor, etc.) to determine a weight, height, length, temperature, heart rate, or other morphological features of the subject. The floor plate 124 can be operatively connected to the computer 104 such that data provided by the floor plate 124 can be stored and/or transmitted by the computer 104.

The apparatus 100 can be configured to release one or more attractants. In some examples, the apparatus 100 is configured to release the attractants at selected time intervals. In other examples, a remote operator can elect to release an attractant using the computer 104/control unit 114. The attractant(s) can be, for example, a physical attractant such as an edible substance, an enticing smell, or an attractive sound.

Physical attractants, such as edible substances or smell-producing materials, can be stored in one or more attractant reservoirs 116a, and can be distributed from the attractant reservoirs using one or more administration devices 122, such as spray nozzles, augers, screw drives, drippers, pumps, etc. In some examples, the chamber 102 can include a bowl or dish 126 into which an attractant can be deposited. Some exemplary edible substances include, but are not limited to, solid pellets, which can be, for example, delivered into the chamber 102 from the attractant reservoir 116a using an auger; a solid attractant stick which can be advanced into the chamber 102 using, for example, a screw drive; and/or a gel, paste, or liquid (e.g., simulated nectar for bats), which can be delivered from a reservoir using, for example, an administration device 122 such as a vaporizing sprayer, dripper, or pump. Enticing smells (e.g., smells that mimic a subject's food, prey, or mate) can be released at selected intervals from the one or more attractant reservoirs 116a, for example, using an administration device 122, such as a vaporizing sprayer, dripper, or pump. Attractive sounds (e.g., prey or mating sounds) can be emitted within the chamber at selected intervals using, for example, an audio speaker and/or animal call. In some examples, the administration devices 122 can be used to administer both attractants and substances.

The one or more imaging devices 106 can be configured to communicate with the computer 104 and to transmit image data and/or other data related to a subject within the chamber 102 to the computer 104. The chamber 102 can be illuminated using, for example, lights 128 (FIG. 2) (e.g., LED lights), in the visible, infrared (IR), and/or ultraviolet (UV) spectrum. The computer 104 can be configured to identify (e.g., using an object detection and classification algorithm such as neural network, convolutional neural network, or other image recognition algorithm) whether the subject within the chamber is a target subject, a member of a target species, an already-marked target subject, and/or a member of a non-target species. In some examples, the algorithms can be trained to assign individual subject animals to groups (e.g., species, population, type) and/or to recognize individual animals using naturally occurring distinctive markings or morphological features (e.g., distance of eyes, muzzle shape, fur coloration, etc.), or identifying marking substances/identifiers previously applied by the apparatus 100 (e.g., paint, dyes, tags such as PIT tags, etc.).

In some examples, such as the example illustrated in FIGS. 1-2, the apparatus 100 can comprise first, second, third, and fourth imaging devices 106a, 106b, 106c, and 106d. The first imaging device can be disposed on the first wall 118a of the chamber 102, the second imaging device 106b on the second wall 118b, the third imaging device 106c on the third wall 118c, and the fourth imaging device 106d on the ceiling 118d (see FIG. 2). This allows images to be captured of the subject head-on (e.g., by the second imaging device 106b) as well as from either side (e.g., by the first and third imaging devices 106a, 106c), and top-down (e.g., by the fourth imaging device 106d).

The multiple imaging devices 106 allow the computer 104 to determine the orientation of the subject within the chamber 104, e.g., which direction the subject is facing. This advantageously allows the computer 104 to wait and administer the substance when the subject is in a desired orientation. For example, some substances, such as vaccines, are best administered into the nasal passages of a subject. The computer's algorithm can automatically determine when the subject is positioned with its nose adjacent a selected administration device 122, such as a spray nozzle, and use the control unit 114 to activate the spray nozzle and administer the substance. In some examples, a remote human operator can manually target a subject using, for example, a live feed from the multiple imaging devices 106. The remote operator can wait until the subject is positioned in the desired orientation (e.g., with its nose adjacent a selected administration device 122) and remotely activate the selected administration device 122. In some examples, the attractant can be disposed at a specific location to encourage or compel a desired orientation, for example, by depositing the attractant in bowl or dish 126 which can be disposed adjacent a selected administration device 122.

The image data and/or video data gathered by the one or more imaging devices 106 can be recorded and/or stored using a memory device either on-board the apparatus 100 (such as an SD flashcard) or remotely (e.g., by transmitting the images/video to a remote device such as a remote computer or cloud-based storage system). Visits by target subjects can be recorded, along with subject data gathered by the imaging devices 106 and/or floor plate 124 (e.g., the length, height, weight, temperature, heart rate, etc. of the subject). This subject data (along with data recording the visit itself) can be stored on-board the apparatus 100 or transmitted to a remote device. In some examples, the transmission of data can be done using a cellular or satellite link. Recording and reporting visits by target subjects allows the quantity of substance delivered to target subjects to be tracked and progress toward goals to be assessed (e.g., progress toward vaccinating and/or marking a critical number of subjects). Recording and reporting subjects' data allows the health of the target species to be assessed.

The apparatus 100 can be configured to provide a deterrent to discourage non-target subjects (or target subjects that have already visited the apparatus 100) from entering and/or remaining inside the chamber 102. Deterrents can include, but are not limited to, mild electrical shocks, bright flashes of light (e.g., using the lights 128 mentioned herein), unpleasant smells, frightening sounds (e.g., using the audio speaker and/or animal call mentioned herein), etc. In some examples, the floor plate 124 can be configured to administer a mild electrical shock as a deterrent. For example, the floor plate 124 can be a metal floor plate comprising a capacitor electrically coupled to a power source.

Once a target subject has been identified and recorded by the computer 104, a substance can be administered to the target subject. Selected options of the one or more reservoirs 116 can be coupled to one or more administration devices 122, such as spray nozzles and/or administration tubes, aiming into the chamber 102. In some examples, the administration devices 122 can be used to administer both attractants and substances.

Substances can be administered to the target subject, in one or more of the following non-limiting, exemplary ways: (1) as an edible pellet dispensed from one or more hoppers/reservoirs through an administration device, such as an administration tube using an auger, (2) as an edible paste, gel, or liquid dispensed from one or more reservoirs 116 using a pump or spray nozzle, (3) as an edible solid stick extending into the chamber 102 and advanced by a screw drive, (4) as a topical solution delivered from one or more reservoirs 116 as a spray or stream via spray nozzles. Because it is often desirable to deliver an edible and a topical substance to the same subject (e.g., to prevent wasting edible substances on a return visitor by marking them as having previously visited) a time lag can be set between delivery of the edible and topical substances to allow for ingestion of the edible substance prior to application of the topical substance (e.g., an identifying marker), in the event that application of the topical substance frightens the subject.

In some examples, rather than using a topical substance for marking, the subject can be marked using a Passive Integrated Transponder (PIT) tag. A PIT tag is a tracking tag that does not require a power source to function, rather, it has an internal microchip that is activated when it passes close to a PIT tag reader. The apparatus 100 can comprise a PIT tag reader that can be operatively coupled to the computer 104 to identify selected subjects that have a PIT tag. In some examples, the apparatus 100 can be used to capture a target subject (e.g., by using a gate to retain the subject with the chamber 102) allowing an operator to manually inject the PIT tag into the subject (e.g., using a needle).

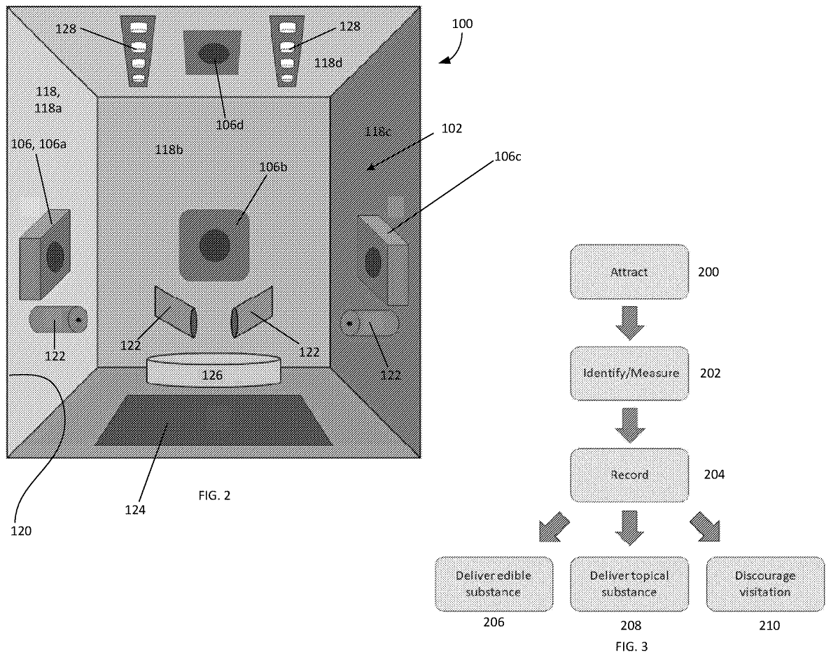

Referring to FIG. 3, use of the administration apparatus 100 can proceed in the following exemplary manner. At 200, the administration apparatus can administer an attractant to attract a subject to the apparatus 100 (e.g., into the chamber 102). At 202, the apparatus 100 can then identify whether the subject is a target subject or a non-target subject using the computer 104 (e.g., using an object detection and classification algorithm such as a neural network, a convolutional neural network, and/or other image recognition algorithm). In some examples, the apparatus 100 can also collect data related to the subject, for example, weight, temperature, heart rate, markings, etc. At 204, these measurements can be stored/recorded on-board the apparatus 100 (such as in a data storage device) or transmitted to a remote device. If the subject is determined to be a target subject, the computer 104 (using the control unit 114) can do one or both of: (1) administer an edible substance (at 206), and/or (2) administer a topical substance. The substance(s) can be applied from the one or more reservoirs 116, using the one or more administration devices 122. If, at 210, the subject is determined to be a non-target subject, the computer 104 (using control unit 114) can discourage visitation by applying a deterrent.

As mentioned previously, in some examples, the computer 104 can be configured to receive data from and/or transmit data to a remote device. In such examples, the remote device can be configured to store data from, send data to, and/or remotely control the administration apparatus 100. The remote device can be, for example, a general-purpose computer, a hand-held mobile device (e.g., a cell phone or tablet), and/or any type of accessory therefore (e.g., a “smart watch” etc.).

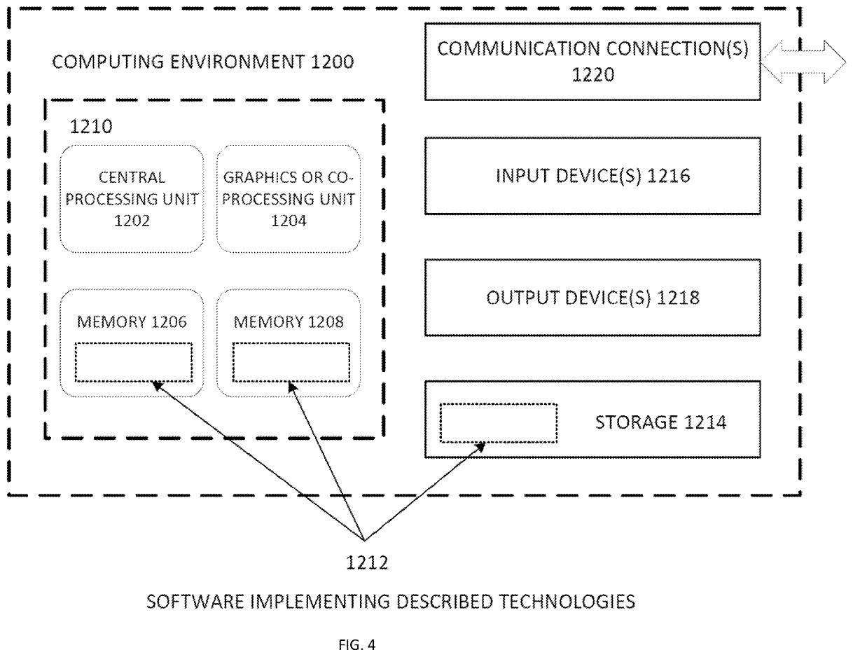

The following is a general description of a computing environment suitable for use with the disclosed control unit 100. FIG. 4 depicts a generalized example of a suitable computing environment 1200 in which software and control algorithms for the described innovations may be implemented. The computing environment 1200 is not intended to suggest any limitation as to scope of use or functionality, as the innovations may be implemented in diverse general-purpose or special-purpose computing systems. In some examples, the computing environment can be or include a processor such as a CPU or a system-on-a-chip (SOC). In other examples, the computing environment 1200 can be any of a variety of computing devices (e.g., computer (such as a laptop or tablet computer), a gaming system, a mobile device, programmable automation controller, etc.).

With reference to FIG. 4, the computing environment 1200 includes one or more processing units 1202, 1204 and memory 1206, 1208 (e.g., for storing system input data). In FIG. 4, this basic configuration 1210 is included within a dashed line. The processing units 1202, 1204 execute computer executable instructions. A processing unit can be or include a general-purpose central processing unit (CPU), a graphics processing unit (GPU), a programmable microcontroller, a processor in an application-specific integrated circuit (ASIC), or any other type of processor. In a multi-processing system, multiple processing units execute computer-executable instructions to increase processing power. For example, FIG. 4 shows a central processing unit 1202 as well as a graphics processing unit 1204. The tangible memory 1206, 1208 can be volatile memory (e.g., registers, cache, RAM), non-volatile memory (e.g., ROM, EEPROM, flash memory, etc.) or some combination of the two, accessible by the processing unit(s). The memory 1206, 1208 stores software 1212 implementing one or more innovations described herein, in the form of computer-executable instructions suitable for execution by the processing unit(s).

A computing system may have additional features. For example, in some embodiments, the computing environment 1200 includes storage 1214, one or more input devices 1216, one or more output devices 1218, and one or more communication connections 1220. An interconnection mechanism (not shown) such as a bus, controller, or network, interconnects the components of the computing environment 1200. Typically, operating system software (not shown) provides an operating environment for other software executing in the computing environment 1200, and coordinates activities of the components of the computing environment 1200. In some embodiments, the computing system can include virtual network computing (VNC) functionality configured to allow operators to access the control unit 200 and/or computing environment 1200 from a remote location. For example, the computing environment 1200 can have remote networking capability (e.g., via satellite modem etc.). The VNC functionality can allow an operator to remotely access the computing environment to, for example, perform maintenance or live monitoring of the administration apparatus 100, or to train an operator on the use of the administration apparatus 100.

The tangible storage 1214 may be removable or non-removable, and includes magnetic disks, secure digital cards (SD Cards), magnetic tapes or cassettes, CD-ROMs, DVDs, or any other medium that can be used to store information in a non-transitory way and can be accessed within the computing environment 1200. The storage 1214 stores instructions for the software 1212 implementing one or more innovations described herein (e.g., for storing image data, subject data such as temperature or weight, location, date, etc.). In some embodiments, the storage can be a “cloud-based” system configured to store data, allow access to data, and/or generate reports. For example, data logs can be sent to a cloud system and reports can be generated therefrom. Users (including, for example, researchers) can access the cloud system remotely through using selected log-in credentials.

The input device(s) 1216 can be, for example: a touch input device, such as a touchscreen display, keyboard, mouse, pen, or trackball; a voice input device; a scanning device; any of various sensors (e.g., the quantity indicator, speed indicator, location unit, etc.); another device that provides input to the computing environment; or combinations thereof. The input device(s) can be remote from the control unit. The output device(s) 1218 can be a display, USB drive (“thumb drive”), SD Card, printer, speaker, CD-writer, transmitter, or another device that provides output from the computing environment 1200.

The communication connection(s) 1220 can enable communication over a communication medium to another computing entity. For example, the communication connection(s) can enable communication between the control unit 200 and a remote input device, for example, a phone app, or a computer browser. The communication medium conveys information, such as computer-executable instructions or other data in a modulated data signal. A modulated data signal is a signal that has one or more of its characteristics set or changed in such a manner as to encode information in the signal. By way of example, and not limitation, communication media can use an electrical, optical, RF, Wi-Fi, Bluetooth, or other carrier.

Any of the disclosed methods can be implemented as computer-executable instructions stored on one or more computer-readable storage media (e.g., one or more optical media discs, volatile memory components (such as DRAM or SRAM), or nonvolatile memory components (such as flash memory or hard drives)) and executed on a computer (e.g., any commercially available computer, including smart phones, other mobile devices that include computing hardware, or programmable automation controllers). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in Python, C, C++, Java, Perl, JavaScript, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communications means include, for example, the Internet, an intranet, software applications, cable (including fiber optic cable), magnetic communications, electromagnetic communications (including RF, microwave, and infrared communications), electronic communications, or other such communication means.

As mentioned previously, in some examples, the remote device can include an application, or “app,” configured to control the administration process and/or track information relating to the administration process and/or the subjects thereof. In some embodiments, the computer 104 can transmit real-time information (e.g., a live camera feed) to the remote device which can be displayed by the app. In some embodiments, multiple computers 104 from multiple apparatuses 100 can transmit real-time information to the same remote device, such that an operator can monitor various apparatuses.

### Overview of Neural Networks

As mentioned above, the computer 104 can implement one or more machine learning algorithms, such as object detection and classification algorithms (e.g., image recognition algorithms, neural networks, etc.) to identify whether a subject within the apparatus 100 is a target subject, a member of a target species, an already-marked target subject, and/or a member of a non-target species. In a computer system, machine learning uses statistical techniques to extract features from a set of training data (e.g., supplied images of target and/or non-target species). The extracted features can then be applied when classifying new data (e.g., images of a not-yet identified subject within the apparatus). Machine learning techniques can be useful in a large number of usage scenarios, such as recognizing images, analyzing and classifying information, and performing various other classification tasks. For example, features can be extracted by training an artificial neural network. After the neural network is trained, new data can be classified according to the trained neural network.

Neural networks implement a computational approach based to some extent on the central nervous systems of animals. Neural networks are composed of interconnected “neurons” that make decisions based on input value(s) and threshold(s). Convolutional neural networks are a class of neural networks that typically involve three stages of computation—convolutional layer(s), fully connected layer(s), and classifier(s).

Neural networks can be used in machine learning and are an example of an artificial-intelligence-based approach to machine learning (as opposed to a modeling-based approach in which a model is specified and various parameters and features of the model are learned). As an example, a neural network can be used to perform image or object recognition. An input image can be converted to an input vector of image pixel values. In a fully connected neural network, each of the pixel values in the input vector is provided to each neuron. A non-linear function is applied to the pixel values at each neuron, and each neuron outputs a value by comparing the result of the non-linear function to the one or more thresholds. The output values from the neurons form an output vector.

A convolutional neural network is a type of neural network in which the neurons have partial connectivity in a particular manner (“convolutional connectivity”). In a convolutional neural network, a two-dimensional (2D) matrix can be used as input. Deep neural networks have multiple layers, which adds richness to the parameters and thresholds of the neurons, classifiers, and other components of the deep neural networks. Each layer can have a different type of connectivity. Individual layers can include convolutional weighting, non-linear transformation, response normalization, and/or spatial pooling.

Typical machine-learning applications operate in two stages. First is the training stage, which involves inputting numerous training images (e.g., images of target species and non-target species). The second stage, which is called the testing stage, on the other hand, typically uses a small amount of input (e.g., image data) and produces a small output (e.g., labels). Convolutional neural networks used in a machine learning context also involve training and testing.

A neural network is typically trained to determine neuron thresholds and classifier model parameters. Input data and available classifier output labels are provided to a training algorithm which attempts to minimize output error over all classifier output labels. Parameter values and thresholds are found that result in the minimum achievable error.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An administration apparatus, comprising: a computer configured to receive data related to a subject adjacent the computer, the computer configured to identify whether the data indicates the presence of either a target subject or a non-target subject; a floorplate operatively coupled to the computer; an administration device configured to administer an attractant and/or a substance when directed by the computer; and a reservoir coupled to the administration device; wherein (i) the apparatus is configured to administer a substance to the target subject when the computer identifies the target subject; and (ii) the apparatus is configured to provide a deterrent to compel the non-target subject to move away from the apparatus when the computer identifies the non-target subject.
Example 2. The apparatus of any example herein, particularly example 1, further comprising one or more imaging devices configured to provide image data to the computer, wherein the computer identifies whether the subject is a target subject or a non-target subject based on the image data by using a neural network.
Example 3. The apparatus of any example herein, particularly any one of examples 1-2, wherein the floorplate is configured to administer a mild electric shock as the deterrent.
Example 4. The apparatus of any example herein, particularly any one of examples 1-3, further comprising: (i) a scale; (ii) a thermometer; (iii) a heart rate monitor; or (iv) any combination of two or more of (i)-(iii).
Example 5. The apparatus of any example herein, particularly any one of examples 1-4, further comprising a sensor configured to detect a presence of the target subject or the non-target subject, and wherein the computer is in a sleep mode or an idle mode prior to the detection of the target subject or the non-target subject.
Example 6. The apparatus of any example herein, particularly any one of examples 1-5, wherein the apparatus is configured to administer an attractant at a selected time interval.
Example 7. The apparatus of any example herein, particularly example 6, wherein the attractant comprises: (i) an edible substance; (ii) an enticing smell; (iii) an attractive sound; or (iv) any combination of two or more of (i)-(iii).
Example 8. The apparatus of any example herein, particularly any one of examples 1-7, wherein the deterrent comprises: (i) an electrical shock; (ii) a flash of light; (iii) an unpleasant smell; (iv) a frightening sound; or (iv) any combination of two or more of (i)-(iv).
Example 9. The apparatus of any example herein, particularly any one of examples 1-8, wherein the substance comprises: (i) an identifying marking; (ii) a medicaments; (iii) a poison; or (iv) any combination of two or more of (i)-(iii).
Example 10. An administration apparatus, comprising: a housing defining a chamber having an opening; an imaging device configured to provide image data showing an interior of the chamber; a computer configured to receive the image data from the imaging device, the computer configured to identify whether the image data shows a target subject or a non-target subject within the chamber; and a reservoir coupled to an administration device configured to administer an attractant and/or a substance to the interior of the chamber when directed by the computer; wherein (i) the administration apparatus is configured to administer the attractant at a selected time interval; (ii) the apparatus is configured to administer the substance when the computer identifies the target subject; and (iii) the apparatus is configured to provide a deterrent such that the non-target subject is compelled to move away from the apparatus when the computer identifies a non-target subject.
Example 11. The apparatus of any example herein, particularly example 10, wherein the computer is disposed within the housing.
Example 12. The apparatus of any example herein, particularly example 10, wherein the computer is a remote computer located remotely to the housing and the imaging device transmits image date to the remote computer.
Example 13. The apparatus of any example herein, particularly any one of examples 10-12, wherein the chamber comprises a floorplate positioned such that a subject within the chamber stands on the floorplate, and wherein the floorplate is configured to send data relating to the subject to the computer.
Example 14. The apparatus of any example herein, particularly any one of examples 10-13, wherein the housing further comprises a gate configured to selectively block the opening to retain the target subject within the chamber.
Example 15. The apparatus of any example herein, particularly any one of examples 10-14, wherein the one or more administration devices comprise: (i) a spray nozzle; (ii) a dripper; (iii) a pump; or (iv) any combination of two or more of (i)-(iii).
Example 16. The apparatus of any example herein, particularly any one of examples 10-15, wherein the substance is a topical substance.
Example 17. The apparatus of any example herein, particularly any one of examples 10-16, wherein the substance is an edible substance.
Example 18. A method, comprising: attracting a subject to an administration apparatus using an attractant; using a computer to identify whether the subject is a target subject or a non-target subject based on data related to the subject; deterring the subject from approaching the administration apparatus using a deterrent if the subject is a non-target subject; and administering a substance to the target subject using an administration device if the subject is a target subject, wherein each administration device is coupled to a respective reservoir.
Example 19. The method of any example herein, particularly example 18, wherein the data related to the subject is image data provided by an imaging device operatively coupled to the computer and wherein using the computer to identify whether the subject is a target subject or a non-target subject based on the image data includes using a neural network.
Example 20. The method of any example herein, particularly any one of examples 18-19, wherein the one or more administration devices comprise: (i) a spray nozzle; (ii) a dripper; (iii) a pump; or (iv) any combination of two or more of (i)-(iii).

The features described herein with regard to any example can be combined with other features described in any one or more of the other examples, unless otherwise stated.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. An administration apparatus, comprising:
a computer configured to receive data related to a subject adjacent the computer, the computer configured to identify whether the data indicates the presence of either a target subject or a non-target subject;
a floorplate operatively coupled to the computer;
an administration device configured to administer an attractant and/or a substance when directed by the computer; and
a reservoir coupled to the administration device;
wherein (i) the apparatus is configured to administer a substance to the target subject when the computer identifies the target subject; and (ii) the apparatus is configured to provide a deterrent to compel the non-target subject to move away from the apparatus when the computer identifies the non-target subject.

2. The apparatus of claim 1, further comprising one or more imaging devices configured to provide image data to the computer, wherein the computer identifies whether the subject is a target subject or a non-target subject based on the image data by using a neural network.

3. The apparatus of claim 1, wherein the floorplate is configured to administer a mild electric shock as the deterrent.

4. The apparatus of claim 1, further comprising:
(i) a scale;
(ii) a thermometer;
(iii) a heart rate monitor; or
(iv) any combination of two or more of (i)-(iii).

5. The apparatus of claim 1, further comprising a sensor configured to detect a presence of the target subject or the non-target subject, and wherein the computer is in a sleep mode or an idle mode prior to the detection of the target subject or the non-target subject.

6. The apparatus of claim 1, wherein the apparatus is configured to administer an attractant at a selected time interval.

7. The apparatus of claim 6, wherein the attractant comprises:
(i) an edible substance;
(ii) an enticing smell;
(iii) an attractive sound; or
(iv) any combination of two or more of (i)-(iii).

8. The apparatus of claim 1, wherein the deterrent comprises:
(i) an electrical shock;
(ii) a flash of light;
(iii) an unpleasant smell;
(iv) a frightening sound; or
(iv) any combination of two or more of (i)-(iv).

9. The apparatus of claim 1, wherein the substance comprises:
(i) an identifying marking;
(ii) a medicaments;
(iii) a poison; or
(iv) any combination of two or more of (i)-(iii).

10. An administration apparatus, comprising:
a housing defining a chamber having an opening;
an imaging device configured to provide image data showing an interior of the chamber;
a computer configured to receive the image data from the imaging device, the computer configured to identify whether the image data shows a target subject or a non-target subject within the chamber; and
a reservoir coupled to an administration device configured to administer an attractant and/or a substance to the interior of the chamber when directed by the computer;
wherein (i) the administration apparatus is configured to administer the attractant at a selected time interval; (ii) the apparatus is configured to administer the substance when the computer identifies the target subject; and (iii) the apparatus is configured to provide a deterrent such that the non-target subject is compelled to move away from the apparatus when the computer identifies a non-target subject.

11. The apparatus of claim 10, wherein the computer is disposed within the housing.

12. The apparatus of claim 10, wherein the computer is a remote computer located remotely to the housing and the imaging device transmits image date to the remote computer.

13. The apparatus of claim 10, wherein the chamber comprises a floorplate positioned such that a subject within the chamber stands on the floorplate, and wherein the floorplate is configured to send data relating to the subject to the computer.

14. The apparatus of claim 10, wherein the housing further comprises a gate configured to selectively block the opening to retain the target subject within the chamber.

15. The apparatus of claim 10, wherein the one or more administration devices comprise:
(i) a spray nozzle;
(ii) a dripper;
(iii) a pump; or
(iv) any combination of two or more of (i)-(iii).

16. The apparatus of claim 10, wherein the substance is a topical substance.

17. The apparatus of claim 10, wherein the substance is an edible substance.

18. A method, comprising:
attracting a subject to an administration apparatus using an attractant;
using a computer to identify whether the subject is a target subject or a non-target subject based on data related to the subject;
deterring the subject from approaching the administration apparatus using a deterrent if the subject is a non-target subject; and
administering a substance to the target subject using an administration device if the subject is a target subject, wherein each administration device is coupled to a respective reservoir.

19. The method of claim 18, wherein the data related to the subject is image data provided by an imaging device operatively coupled to the computer and wherein using the computer to identify whether the subject is a target subject or a non-target subject based on the image data includes using a neural network.

20. The method of claim 18, wherein the one or more administration devices comprise:
(i) a spray nozzle;
(ii) a dripper;
(iii) a pump; or
(iv) any combination of two or more of (i)-(iii).
